(19) United States
(12) Patent Application Publication
(10) Pub. No.: US 2017/0232255 A1
(43) Pub. Date: Aug. 17, 2017

(54) SYSTEMS AND METHODS FOR SPINAL CORD STIMULATION TRIAL

(71) Applicant: Pacesetter, Inc., Sunnyvale, CA (US)

(72) Inventors: Alexander Kent, Mountain View, CA (US); Yelena Nabutovsky, Mountain View, CA (US); Stuart Rosenberg, Castaic, CA (US); Gene A. Bornzin, Simi Valley, CA (US)

(21) Appl. No.: 15/056,595

(22) Filed: Feb. 29, 2016

Related U.S. Application Data
(60) Provisional application No. 62/294,462, filed on Feb. 12, 2016.

(51) Int. Cl. A61N 1/36; A61N 1/05

(52) U.S. Cl. CPC A61N 1/36017; A61N 1/0551; A61N 1/36021

(57) ABSTRACT

The present disclosure provides a spinal cord stimulation (SCS) trial system. The SCS trial system includes at least one rigid needle lead including a biocompatible conductor extending from a proximal end to a distal end, and insulation surrounding at least a portion of the biocompatible conductor, wherein the at least one rigid needle lead is configured to pierce the skin of a patient and be percutaneously implanted in the patient such that the distal end is proximate to at least one of a dorsal column, a dorsal root, dorsal root ganglia, and a peripheral nerve of the patient. The system further includes an external pulse generator (EPG) coupled to the at least one rigid needle lead and configured to apply electrical stimulation to the patient via the at least one rigid needle lead.

### A. PRIORITY

This application claims the benefit of U.S. Provisional Application No. 62/294,462, entitled Systems and Methods For Spinal Cord Stimulation Trial, filed Feb. 12, 2016, which is incorporated herein by reference in its entirety to provide continuity of disclosure.

### B. FIELD OF THE DISCLOSURE

The present disclosure relates generally to neurostimulation systems, and more particularly to spinal cord stimulation trials.

### C. BACKGROUND ART

Neurostimulation is a treatment method utilized for managing the disabilities associated with pain, movement disorders such as Parkinson's Disease (PD), dystonia, and essential tremor, and also a number of psychological disorders such as depression, mood, anxiety, addiction, and obsessive compulsive disorders.

Neurostimulation systems include spinal cord stimulation (SCS) systems. Before having a permanent SCS system implanted, patients may undergo an SCS trial to determine whether SCS will be successful in reducing pain. However, it is believed that only roughly 20% of chronic pain patients who are indicated for SCS undergo a trial. This may be the result of lack of familiarity of SCS therapy by the treating physician and/or patient apprehension about the invasiveness of the trial.

Further, a relatively low percentage of patients who undergo an SCS trial successfully convert to a permanent SCS system. Reasons for failure include lack of pain relief, lack of paresthesia, and discomfort resulting from stimulation. Further, post-operative pain from the trial may mask SCS-generated improvements in reducing pain. Accordingly, there is a need for an SCS trial system that increases accessibility of SCS therapy and that improves the trial-to-permanent success rate.

### BRIEF SUMMARY OF THE DISCLOSURE

In one embodiment, the present disclosure is directed to a spinal cord stimulation (SCS) trial system. The SCS trial system includes at least one rigid needle lead including a biocompatible conductor extending from a proximal end to a distal end, and insulation surrounding at least a portion of the biocompatible conductor, wherein the at least one rigid needle lead is configured to pierce the skin of a patient and be percutaneously implanted in the patient such that the distal end is proximate to at least one of a dorsal column, a dorsal root, dorsal root ganglia, and a peripheral nerve of the patient. The system further includes an external pulse generator (EPG) coupled to the at least one rigid needle lead and configured to apply electrical stimulation to the patient via the at least one rigid needle lead.

In another embodiment, the present disclosure is directed to a method for implanting a spinal cord stimulation (SCS) trial system in a patient. The method includes percutaneously implanting at least one rigid needle lead by piercing the skin of the patient, the at least one rigid needle lead including a biocompatible conductor extending from a proximal end to a distal end, and insulation surrounding at least a portion of the biocompatible conductor, the at least one rigid needle lead percutaneously implanted such that the distal end is proximate to at least one of a dorsal column, a dorsal root, dorsal root ganglia, and a peripheral nerve of the patient, electrically coupling an external pulse generator (EPG) to the at least one rigid needle lead, and applying electrical stimulation to the patient via the at least one rigid needle lead.

In another embodiment, the present disclosure is directed to a microdriver system for use in orienting and percutaneously implanting at least one rigid needle lead in a patient. The system includes a base configured to be positioned on skin of the patient, an arm coupled to the base and configured to be translated relative to the base, and a mounting plate coupled to the arm and configured to be translated relative to the arm, the mounting plate further configured to attach to the at least one rigid needle lead.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic view of one embodiment of a stimulation system.

FIG. 2 is a schematic diagram of one embodiment of a microdriver system that may be used to implant an SCS trial system.

FIG. 3 is a schematic diagram of an implantation trajectory that may be achieved using the microdriver system shown in FIG. 2.

FIG. 4 is a schematic diagram of multiple needle leads that may be used in an SCS trial system.

FIG. 5 is a schematic diagram of an SCS trial system implanted for a chronic trial.

FIG. 6 is a flow chart of one embodiment of a method for implanting a spinal cord stimulation (SCS) trial system in a patient.

FIG. 7 is a flow chart of one embodiment of a method for coupling an external pulse generator to at least one needle lead.

FIG. 8 is a flow chart of one embodiment of a method for verifying a position of an implanted needle lead.

Corresponding reference characters indicate corresponding parts throughout the several views of the drawings.

### DETAILED DESCRIPTION OF THE DISCLOSURE

The present disclosure provides a spinal cord stimulation (SCS) trial system that may be used to determine the efficacy of SCS on a patient before implantation of a permanent SCS system. The SCS trial system applies stimulation to the spinal cord using one or more minimally invasive needle leads. This facilitates improving the SCS trial experience and success rate by reducing post-operative pain associated with the SCS trial. Using miniaturized leads also facilitates increasing the accessibility of an SCS trial by reducing patient apprehension about the procedure.

Neurostimulation systems are devices that generate electrical pulses and deliver the pulses to nerve tissue of a patient to treat a variety of disorders. Spinal cord stimulation (SCS) is the most common type of neurostimulation within the broader field of neuromodulation. In SCS, electrical pulses are delivered to nerve tissue of the spinal cord for the purpose of chronic pain control. While a precise understanding of the interaction between the applied electrical energy and the nervous tissue is not fully appreciated, it is known that application of an electrical field to spinal nervous tissue can effectively inhibit certain types of pain transmitted from regions of the body associated with the stimulated nerve tissue to the brain. Specifically, applying electrical energy to the spinal cord associated with regions of the body afflicted with chronic pain can induce “paresthesia” (a subjective sensation of numbness or tingling) in the afflicted bodily regions.

SCS systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals (also electrically coupled to the wire conductors) that are adapted to receive electrical pulses. The distal end of a respective stimulation lead is implanted within the epidural space to deliver the electrical pulses to the appropriate nerve tissue within the spinal cord that corresponds to the dermatome(s) in which the patient experiences chronic pain. Stimulation may also be applied to the dorsal root ganglia (DRG) and/or peripheral nerves to reduce pain. The stimulation leads are then tunneled to another location within the patient's body to be electrically connected with a pulse generator or, alternatively, to an “extension.”

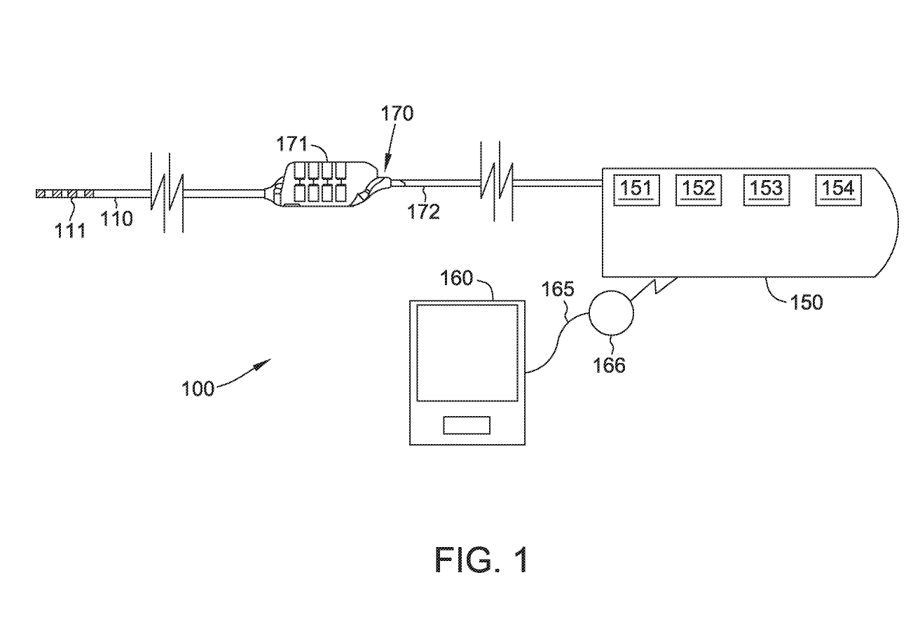

Referring now to the drawings, and in particular to FIG. 1, a stimulation system is indicated generally at 100. Stimulation system 100 generates electrical pulses for application to tissue of a patient, or subject, according to one embodiment. Stimulation system 100 includes an implantable pulse generator (IPG) 150 that is adapted to generate electrical pulses for application to tissue of a patient. Implantable pulse generator 150 typically includes a metallic housing that encloses a controller 151, pulse generating circuitry 152, a battery 153, far-field and/or near field communication circuitry 154, and other appropriate circuitry and components of the device. Controller 151 typically includes a microcontroller or other suitable processor for controlling the various other components of the device. Software code is typically stored in memory of implantable pulse generator 150 for execution by the microcontroller or processor to control the various components of the device.

Implantable pulse generator 150 may comprise one or more attached extension components 170 or be connected to one or more separate extension components 170. Alternatively, one or more stimulation leads 110 may be connected directly to implantable pulse generator 150. Within implantable pulse generator 150, electrical pulses are generated by pulse generating circuitry 152 and are provided to switching circuitry. The switching circuit connects to output wires, traces, lines, or the like (not shown) which are, in turn, electrically coupled to internal conductive wires (not shown) of a lead body 172 of extension component 170. The conductive wires, in turn, are electrically coupled to electrical connectors (e.g., “Bal-Seal” connectors) within connector portion 171 of extension component 170. The terminals of one or more stimulation leads 110 are inserted within connector portion 171 for electrical connection with respective connectors. Thereby, the pulses originating from implantable pulse generator 150 and conducted through the conductors of lead body 172 are provided to stimulation lead 110. The pulses are then conducted through the conductors of stimulation lead 110 and applied to tissue of a patient via electrodes 111. Any suitable known or later developed design may be employed for connector portion 171.

Stimulation lead(s) 110 may include a lead body of insulative material about a plurality of conductors within the material that extend from a proximal end of stimulation lead 110 to its distal end. The conductors electrically couple a plurality of electrodes 111 to a plurality of terminals (not shown) of stimulation lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number of electrodes 111, terminals, and internal conductors. As described in detail below, in the embodiments described herein, stimulation lead 110 is a rigid needle lead formed from a biocompatible conductor with an insulative coating.

A controller device 160 may be implemented to recharge battery 153 of implantable pulse generator 150 (although a separate recharging device could alternatively be employed). A “wand” 165 may be electrically connected to controller device through suitable electrical connectors (not shown). The electrical connectors are electrically connected to a coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The systems and methods described herein provide an SCS trial system that may be used to determine the efficacy of SCS on a patient before implantation of a more permanent SCS system, such as stimulation system 100 (shown in FIG. 1). The SCS trial system described herein applies stimulation to the spinal cord using a minimally invasive needle lead. As used herein, a needle lead refers to a rigid, relatively thin lead that is able to pierce the skin of the patient without the use of any additional surgical instruments (e.g., introducers). This facilitates improving the trial experience and success rate by reducing post-operative pain associated with the SCS trial. Using miniaturized leads also facilitates increasing the accessibility of an SCS trial by decreasing invasiveness and reducing patient apprehension about the procedure. Notably, the systems and methods described herein may be used for pre-trial screening or as an alternative to existing SCS trial systems.

After implantation, the systems and methods described herein are used to apply electrical stimulation to the dorsal column, dorsal root(s), dorsal root ganglia (DRG), or peripheral nerve(s) to determine the effectiveness of SCS or peripheral nerve stimulation (PNS) in treating the patient's pain. The applied electrical stimulation may be burst stimulation, tonic stimulation, high-frequency stimulation, etc. If this testing is successful (e.g., if the testing results in a reduction in pain of 50% or more), then SCS is likely to benefit the patient and the patient could proceed to obtain a known SCS trial system or move directly to a permanent SCS system.

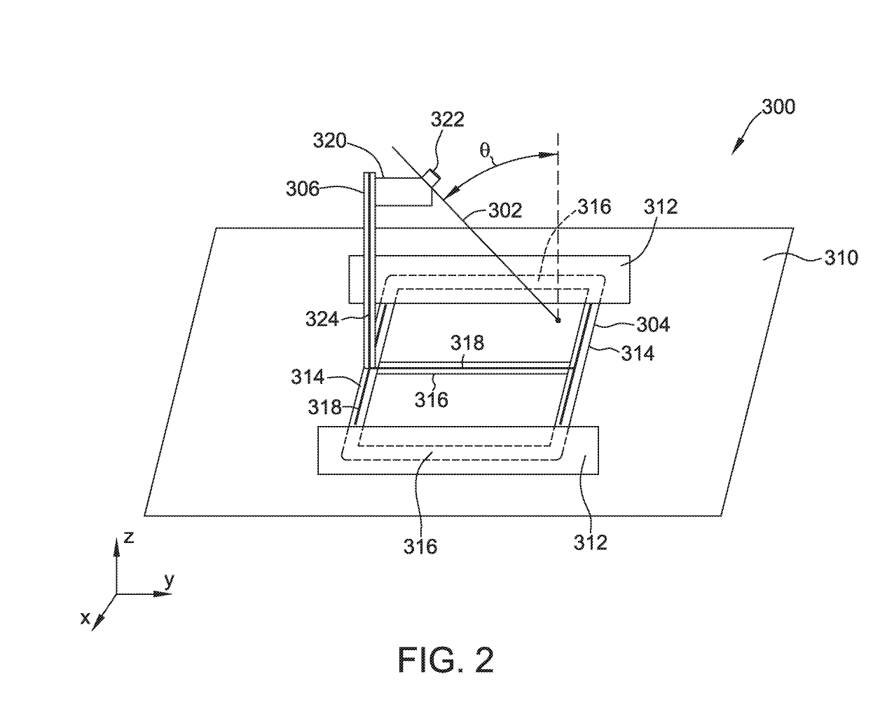

FIG. 2 is a schematic diagram of one embodiment of a microdriver system 300 that may be used to implant an SCS trial system. Microdriver system 300 facilitates delivering an SCS needle lead 302 to a spinal cord target of a patient. Microdriver system 300 includes a base 304 and an arm 306 extending from base 304 in a direction substantially orthogonal to base 304.

As shown in FIG. 2, to deliver SCS needle lead 302, base 304 is attached to the skin 310 of a patient lying in a prone position such that base 304 is substantially flush with skin 310. Base 304 may be attached using one or more adhesive strips 312 (e.g., surgical tape) to the patient's back. The target for insertion is based on patient-reported descriptions of pain location to classify painful dermatomes. This is used to identify the corresponding sensory fibers from these dermatomes within the spinal cord, DRG, dorsal root, or peripheral nerves. Based on this location, the clinical implants SCS needle lead 302 at the appropriate vertebral level, using palpation to find the pedicle or foramen.

In this embodiment, base 304 is substantially in the shape of an “8”. Specifically, base 304 includes two first struts 314 extending along an x-direction (e.g., the medial-lateral direction), and three second struts 316 extending between first struts 314 along a y-direction (e.g., the cranial-caudal direction). Alternatively, base 304 may have any suitable shape.

In this embodiment, base 304 includes one or more tracks 318 that enable arm 306 to translate relative to base 304. Specifically, both first struts 314 include track 318 to translate arm 306 along the x-direction, and one of second struts includes track 318 to translate arm 306 along the y-direction. Arm 306 may be moved manually (e.g., by a human operator), or may be controlled using a suitable electromechanical system.

As shown in FIG. 2, a mounting plate 320 is attached to arm 306. Mounting plate 320 includes a thumb screw attachment 322 that facilitates attaching SCS needle lead 302 to mounting plate 320. In this embodiment, arm 306 includes an arm track 324 that enables mounting plate 320 to be translated along a z-direction (e.g., the anterior-posterior direction). Mounting plate 320 may be moved manually (e.g., by a human operator), or may be controlled using a suitable electromechanical system. Moving mounting plate 320 and arm 306 changes an insertion angle, θ, of SCS needle lead 302. When SCS needle lead 302 is substantially orthogonal to skin 310, θ is approximately 0°. For insertion, using thumb screw attachment 322 and/or manually controlled motors, SCS needle lead 302 is advanced along the current implantation trajectory.

In this embodiment, SCS needle lead 302 is a thin lead (e.g., approximately 0.12 to 0.35 millimeters (mm) in diameter, and approximately 50 mm in length) constructed of a biocompatible conductor (e.g., a platinum-iridium alloy) with an insulative coating (e.g., parylene). One or more electrodes are formed at a distal end of SCS needle lead 302 by exposing portions of biocompatible conductor (e.g., by selectively not including insulative coating over those portions of biocompatible conductor). SCS needle lead 302 is rigid such that SCS needle lead 302 is capable of easily piercing the skin of a patient without using additional surgical instruments.

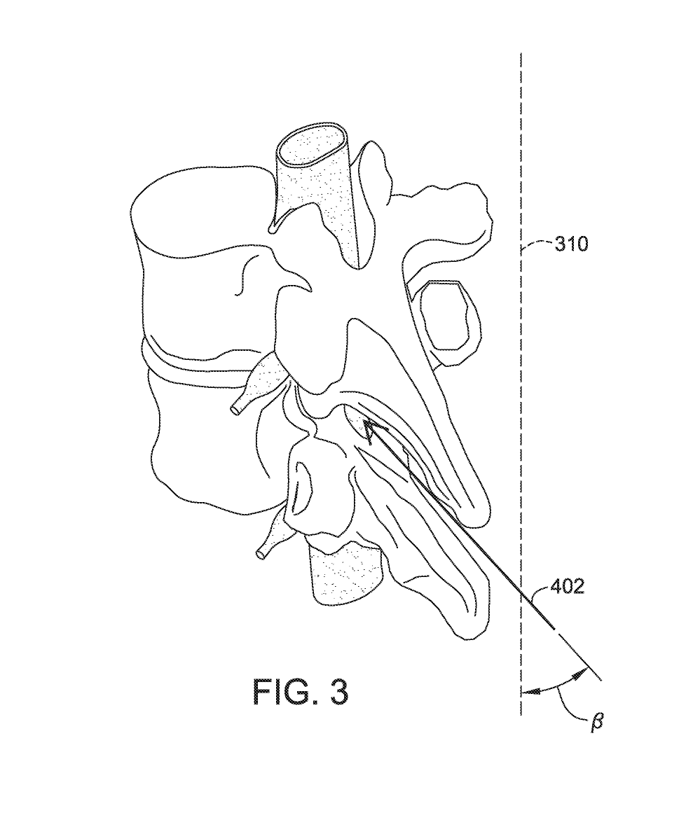

For delivery of electrical stimulation, SCS needle lead 302 is implanted percutaneously near the dorsal column, dorsal roots, or dorsal root ganglia (DRG) of the spinal cord. FIG. 3 is a schematic diagram of an example implantation trajectory 402 for SCS needle lead 302 that avoids other structures (e.g., vertebral bone). During implantation, SCS needle lead 302 pierces skin 310. The rigidity of SCS needle lead 302 allows SCS needle lead 302 to pierce skin 310. As shown in FIG. 3, SCS needle lead 302 is inserted at an angle (i.e., 8 is not equal to 0°). For example, an angle β formed between SCS needle lead 302 and skin may be, for example, between approximately 30° and 45°. An appropriate insertion angle will likely be known by the clinician. By moving arm 306 and mounting plate 320 along tracks 318 and arm track 324, microdriver system 300 enables adjusting the angle of SCS needle lead 302 such that a proper implantation trajectory to advance SCS needle lead 302 along implantation trajectory 402 is achieved.

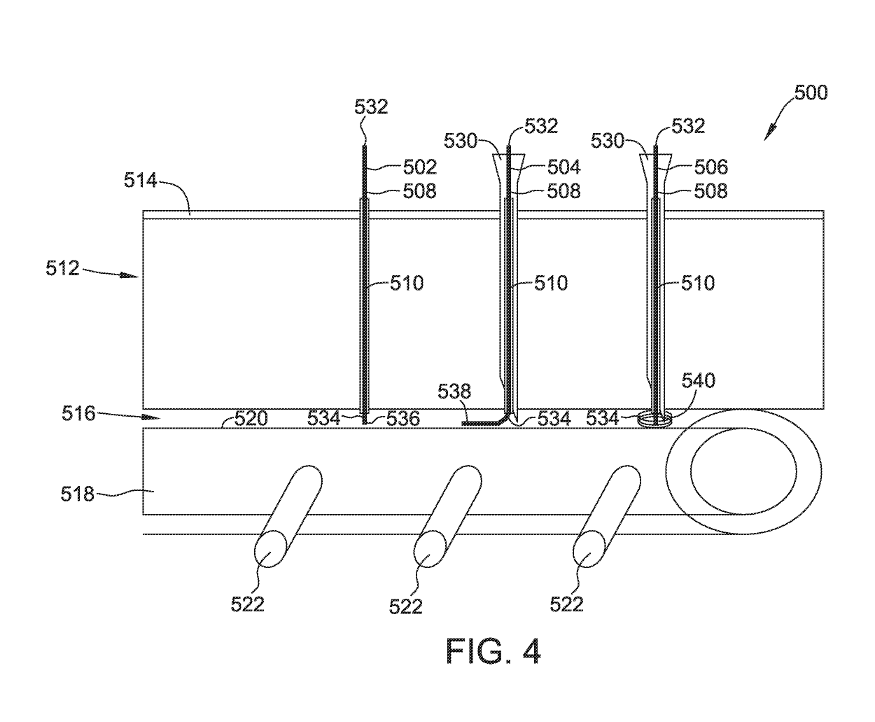

FIG. 4 is a schematic diagram of showing multiple examples of SCS needle leads that may be used in an SCS trial system 500. An SCS system may include one or more SCS needle leads, such as SCS needle lead 302 (shown in FIG. 2), that may be implanted, for example, using microdriver system 300 (also shown in FIG. 2). Further, an SCS system may include a single SCS needle lead, or multiple needle leads. Accordingly, the SCS needle leads shown in FIG. 4 may be used independently of one another or in combination with one another. In FIG. 4, SCS trial system 500 includes a first lead 502, a second lead 504, and a third lead 506. As shown in FIG. 4, each lead 502, 504, 506 includes a conductor 508 and insulation 510 that surrounds at least a portion of conductor 508. Specifically, each lead 502, 504, 506 includes sufficient insulation 510 such that conductor 508 is insulated from muscle tissue 512 of the patient when implanted.

As shown in FIG. 4, after percutaneous implantation, each lead 502, 504, 506 extends through skin 514 and muscle tissue 512 to reach the epidural space 516 between muscle tissue 512 and the spinal cord 518 of the patient. Spinal cord 518 includes the dura layer 520, and dorsal roots 522 extend from spinal cord 518.

Leads 502, 504, and 506 may have the same or different configurations from each other. For example, in this embodiment, second and third leads 504 and 506 include a cannula 530. Each lead 502, 504, and 506 includes a proximal end 532 and an opposite distal end 534. In this embodiment, at distal end 534, first lead 502 has a straight tip 536, second lead 504 has a curved tip 538, and third lead 506 has a spiral tip 540. After implantation, second lead 504 may be rotated to achieve a desired orientation of curved tip 538. In some embodiments, proximal end 532 of second lead 504 includes a marker (e.g., indicia) that may be used to determine the orientation of curved tip 538. Relative to straight tip 536, curved and spiral tips 538 and 540 increase the electrode surface area for stimulation of spinal cord 518.

Tips 536, 538, and 540 include one or more stimulating electrodes, and may be constructed from a shape memory material and/or a superelastic material (e.g., nitinol) to conform between different shapes (e.g., straight to curved). As shown in FIG. 4, exposed (i.e., non-insulated) portions of conductor 508 form the electrodes. In certain embodiments, the change in shape may occur due to the shape memory of the material, such that a change in temperature above the transformation temperature of the material (e.g., a change from room temperature to body temperature after a lead has been implanted) may affect a change in the shape of the material. In certain embodiments, the change in shape may occur due to the superelasticity of the material, without requiring a change of temperature of the material to recover to an undeformed shape. For example, in certain embodiments tips 536, 538, and 540 are constructed from nitinol, and tips 536, 538, and 540 can be bent and returned to their original shapes without requiring a change in temperature, due to the superelasticity of nitinol. In certain embodiments, the change in shape may be due to both the shape memory (i.e., change in temperature) and superelasticity of the material. Cannulas 530 may be, for example, 22 to 28 gauge, and may be used to maintain tips 538 and 540 in a straight orientation during implantation, until the spinal cord target is reached, at which point cannula 530 may be retracted.

During implantation, test stimulation or impedance measurements may be used to determine a current location of leads 502, 504, 506. In one embodiment, with every advancement step (e.g., 1.0 mm) of a lead towards the spinal cord, low amplitude tonic stimulation is delivered to evaluate whether the lead is nearing the spinal cord. If the patient feels paresthesia, then the lead is sufficient close to generate a symptomatic response. In another embodiment, electrical impedance (Z) is measured by applying a current (I), measuring a resulting voltage (V), and calculating the Z=V/I. As the lead is advanced through the back musculature (resistivity of approximately 230 ohm-centimeters (Ω-cm)) and into the epidural fat (resistivity of approximately 2300 Ω-cm), the impedance increases substantially. In general the impedance values of leads in the systems and methods described herein may be approximately 50% of those measured with known SCS leads. Thus, as described above, test stimulation and impedance measurements may be used to determine a location of a lead as it approaches the spinal cord.

Further, if leads 502, 504, 506 are implanted chronically, their position may be monitored to ensure they remain in the same place after implantation, and do not shift position. Impedance measurements may be used as described above. In an alternative embodiment, a photoelectric diffuse sensor is used to verify lead position. The photoelectric diffuse sensor may include, for example, a lighting device at the tip that emits light (e.g., pulsed, infrared, visible red, and/or laser light). The emitted light is reflected off an anatomical structure and returns to the tip, where it is measured by a sensor. By measuring the returning light, the proximity of the tip and to the anatomical structure can be determined, and the position of the lead may be verified by determining the proximity of the lead tip to the anatomical structure.

In another alternative embodiment, neural activity (e.g., evoked compound action potential (ECAP)) may be recorded, for example, using the same tip electrode used to deliver stimulation. That is, after applying stimulation using the tip electrode, a peak to peak voltage may be measured using the tip electrode. In general, ECAP increases as the electrode moves closer to an anatomical structure, and decreases as the electrode moves away from the anatomical structure. Accordingly, similar to the optical sensor, the neural activity may be recorded and analyzed to verified lead position by determining that a distance to an anatomical structure remains unchanged.

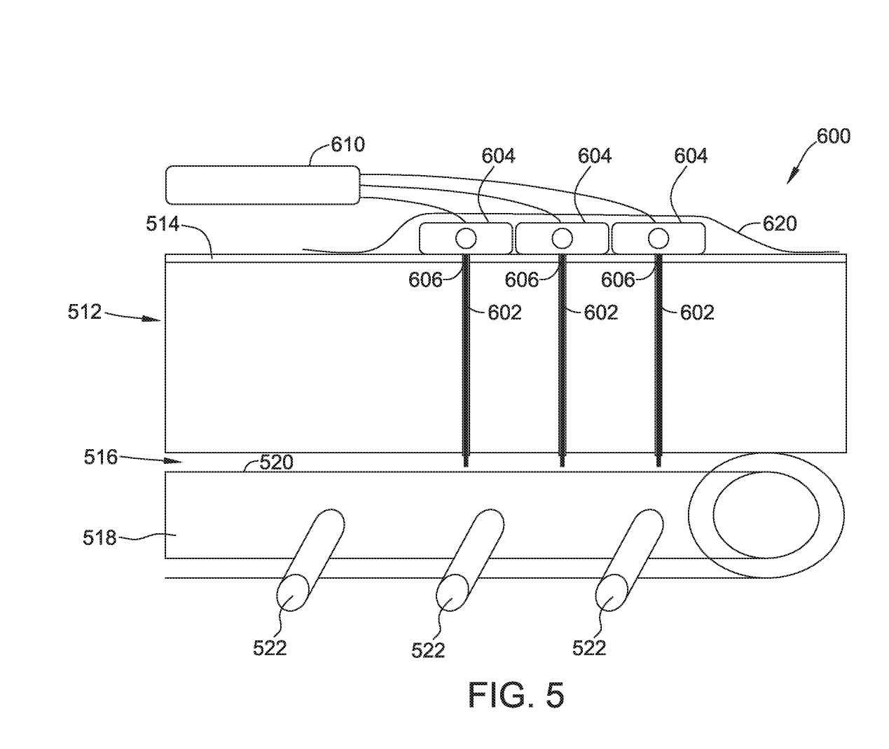

Leads 502, 504, 506 may be implanted for either an acute or chronic trial. An acute trial may be an on-table procedure that only lasts a few minutes, while a chronic trial may last much longer (e.g., a few days). FIG. 5 is a schematic diagram of an SCS trial system 600 implanted for a chronic trial. As shown in FIG. 5, for a chronic trial, while microdriver system 300 is still attached, each lead 602 is crimped and a button connector 604 is attached to a proximal end 606 of lead 602. Button connectors 604 facilitate ensuring leads 602 do not move. In some embodiments, button connectors 604 elute topical anesthetic via a controlled-release coating to reduce pain associated with the chronic implant.

Button connectors 604 are then electrically connected to an external pulse generator (EPG) 610. EPG 610 controls electrical stimulation delivered by leads 602. In some embodiments, EPG 610 may also be used for an acute trial, with suitable adhesive (e.g., tape) used to secure EPG 610. Although SCS trial system 600 includes three leads 602 in this embodiment, alternatively, SCS trial system 600 may include any suitable number of leads, including one lead. To facilitate reducing infection, button connectors 604 are covered by a water-proof patch 620 that adheres to the patient's skin 514.

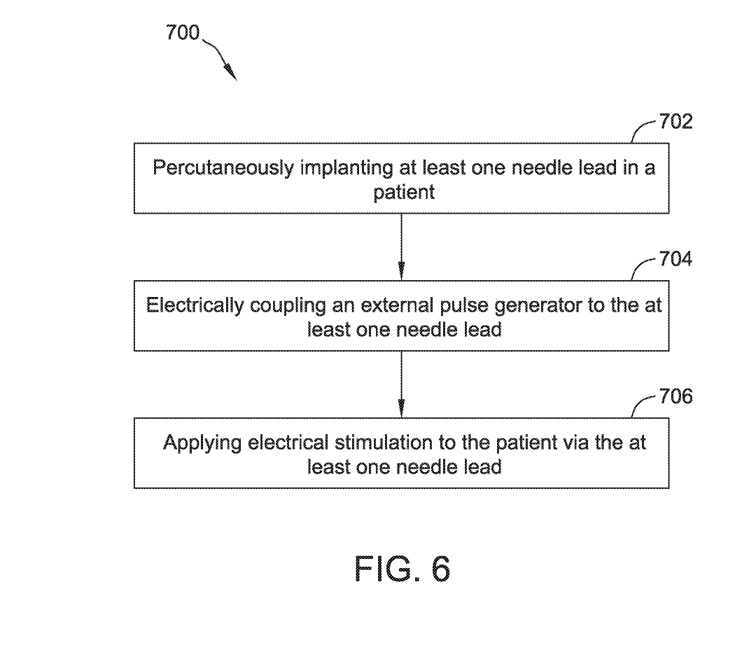

FIG. 6 is a flow chart of one embodiment of a method 700 for implanting a spinal cord stimulation (SCS) trial system in a patient. Method 700 includes percutaneously implanting 702 at least one needle lead. In this embodiment, implanting 702 the at least one needle lead includes piercing the skin of the patient using the at least one needle lead. In this embodiment, the at least one needle lead includes a biocompatible conductor extending from a proximal end to a distal end, and insulation surrounding at least a portion of the biocompatible conductor. The at least one needle lead is percutaneously implanted 702 such that the distal end is proximate to at least one of a dorsal column, a dorsal root, dorsal root ganglia, and a peripheral nerve of the patient. Method 700 further includes electrically coupling 704 an external pulse generator (EPG) to the at least one needle lead. Method 700 further includes applying 706 electrical stimulation to the patient via the at least one needle lead.

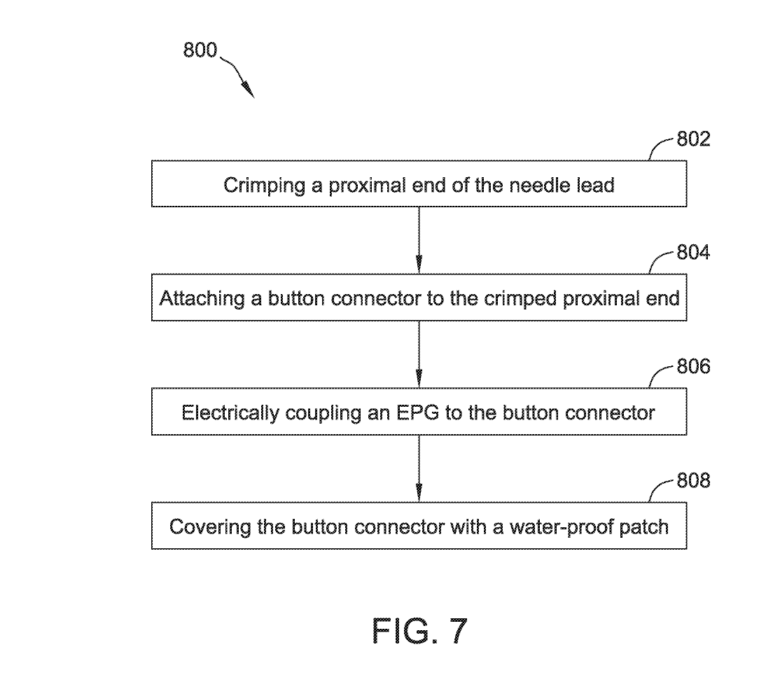

FIG. 7 is a flow chart of one embodiment of a method 800 for coupling an EPG, such as EPG 610 (shown in FIG. 5), to at least one needle lead. Method 800 may be used to implement, for example, coupling 704 (shown in FIG. 6). Method 800 includes crimping 802 a proximal end of the at least one needle lead. A button connector is attached 804 to the crimped proximal end. The button connector may include, for example, button connector 604 (shown in FIG. 5). The button connector facilitates maintaining a position of the attached needle lead. Further, the button connector may elute a topical anesthetic to reduce pain. Method 800 further includes electrically coupling 806 the EPG to the button connector. This in turn electrically couples the EPG to the at least one needle lead. To reduce infection, the button connector may be covered 808 with a water-proof patch that adheres to the patient's skin.

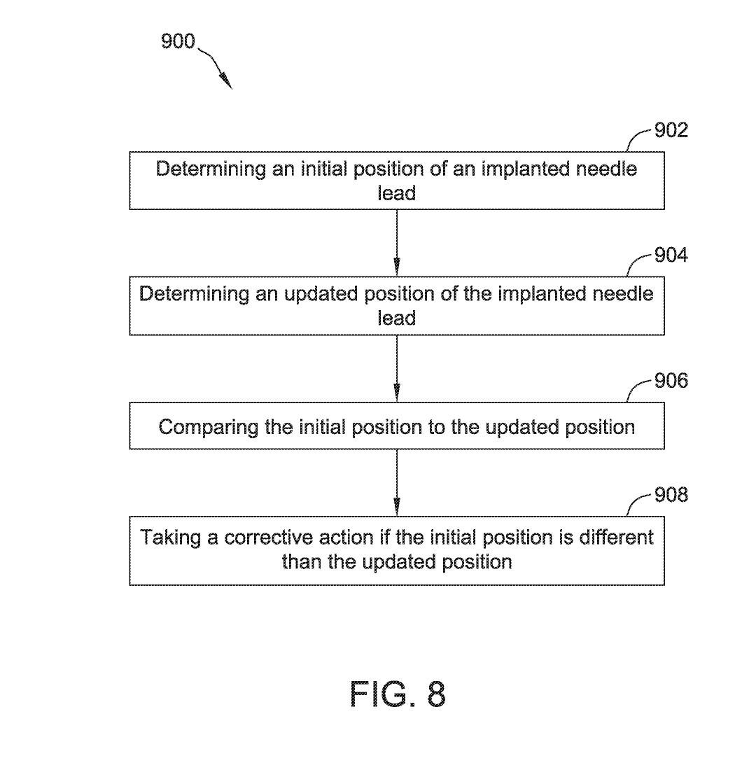

FIG. 8 is a flow chart of one embodiment of a method 900 for verifying a position of an implanted needle lead. Method 900 includes determining 902 an initial position of the implanted needle lead (e.g., at the time of implantation). At a later time, an updated position of the implanted needle lead is determined 904. The initial and updated positions may be determined for example, using impedance measurements, using a photoelectric sensor, and/or using neural activity measurements, as described above. Method 900 further includes comparing 906 the initial position to the updated position. If the initial position matches the updated position, the implanted needle lead has not shifted. However, if the initial position is different than the updated position, then the needle lead has likely shifted, an appropriate corrective action (e.g., surgically adjusting the lead position, ceasing stimulation, etc.) is taken 908.

With leads implanted for either an acute or chronic trial, electrical stimulation may be delivered in various ways, including bipolar and monopolar configurations. For bipolar stimulation, each needle lead may contain two or more electrode contacts at the tip (e.g., formed by selectively exposing portions of the conductor). The electrode contacts may be arranged in series along a length of the tip, such that a stimulation location may be selected accordingly. Alternatively, two electrodes could be placed at the most distal portion of the tip in a concentric arrangement.

Monopolar stimulation may be delivered using one or more electrode contacts at the tip of the lead and a counter electrode. The counter electrode could be base 304 (for acute implants) or button connectors 604 (for chronic implants). These configurations could be used for test stimulation and impedance measurements during lead advancement (as described above), as well during therapeutic stimulation delivered to the target location of the spinal cord.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A spinal cord stimulation (SCS) trial system comprising:
at least one rigid needle lead comprising:
a biocompatible conductor extending from a proximal end to a distal end; and
insulation surrounding at least a portion of the biocompatible conductor, wherein the at least one rigid needle lead is configured to pierce the skin of a patient and be percutaneously implanted in the patient such that the distal end is proximate to at least one of a dorsal column, a dorsal root, dorsal root ganglia, and a peripheral nerve of the patient; and
an external pulse generator (EPG) coupled to the at least one rigid needle lead and configured to apply electrical stimulation to the patient via the at least one rigid needle lead.

2. The SCS trial system of claim 1, wherein the distal end comprises a straight tip.

3. The SCS trial system of claim 1, wherein the distal end comprises a curved tip.

4. The SCS trial system of claim 1, wherein the distal end comprises a spiral tip.

5. The SCS trial system of claim 1, further comprising a button connector attached to the proximal end of the at least one rigid needle lead, the EPG coupled to the button connector.

6. The SCS trial system of claim 5, further comprising a water-proof patch configured to adhere to skin of the patient and cover the button connector.

7. The SCS trial system of claim 1, wherein the at least one rigid needle lead further comprises a cannula surrounding the biocompatible conductor and insulation, the cannula configured to facilitate implantation of the at least one rigid needle lead.

8. The SCS trial system of claim 1, wherein the distal end comprises a tip made of at least one: a shape memory material configured to change shape in response to a change in temperature and a superelastic material configured to recover an undeformed shape without a change in temperature.

9. The SCS trial system of claim 1, further comprising a microdriver system configured to orient and advance the at least one rigid needle lead during implantation, the microdriver system comprising:
a base;
an arm coupled to the base and configured to be translated relative to the base; and
a mounting plate coupled to the arm and configured to be translated relative to the arm, the mounting plate comprising a thumb screw attachment configured to attach the at least one rigid needle lead to the mounting plate.

10. A method for implanting a spinal cord stimulation (SCS) trial system in a patient, the method comprising:
percutaneously implanting at least one rigid needle lead by piercing the skin of the patient, the at least one rigid needle lead including a biocompatible conductor extending from a proximal end to a distal end, and insulation surrounding at least a portion of the biocompatible conductor, the at least one rigid needle lead percutaneously implanted such that the distal end is proximate to at least one of a dorsal column, a dorsal root, dorsal root ganglia, and a peripheral nerve of the patient;
electrically coupling an external pulse generator (EPG) to the at least one rigid needle lead; and
applying electrical stimulation to the patient via the at least one rigid needle lead.

11. The SCS method of claim 10, wherein percutaneously implanting at least one rigid needle lead comprises percutaneously implanting at least one rigid needle lead having a distal end that includes a straight tip, and wherein piercing the skin of the patient comprises piercing the skin of the patient using the straight tip of the rigid needle lead.

12. The SCS method of claim 10, wherein percutaneously implanting at least one rigid needle lead comprises percutaneously implanting at least one rigid needle lead having a distal end that includes a curved tip.

13. The SCS method of claim 10, wherein percutaneously implanting at least one rigid needle lead comprises percutaneously implanting at least one rigid needle lead having a distal end that includes a spiral tip.

14. The SCS method of claim 10, wherein electrically coupling an EPG to the at least one rigid needle lead comprises:
crimping the proximal end of the at least one rigid needle lead;
attaching a button connector to the crimped proximal end; and
electrically coupling the EPG to the button connector.

15. The SCS method of claim 14, further comprising covering the button connector with a water-proof patch.

16. The SCS method of claim 10, wherein percutaneously implanting at least one rigid needle lead comprises percutaneously implanting at least one rigid needle lead using a cannula that surrounds the biocompatible conductor and insulation.

17. The SCS method of claim 10, further comprising determining a location of the at least one rigid needle lead within the patient using at least one of test stimulation, impedance measurements, photoelectric sensor measurements, and neural activity measurements.

18. A microdriver system for use in orienting and percutaneously implanting at least one rigid needle lead in a patient, the microdriver system comprising:
a base configured to be positioned on skin of the patient;
an arm coupled to the base and configured to be translated relative to the base; and
a mounting plate coupled to the arm and configured to be translated relative to the arm, the mounting plate further configured to attach to the at least one rigid needle lead.

19. The microdriver system of claim 18, wherein the mounting plate comprises a thumb screw attachment configured to attach the at least one rigid needle lead to the mounting plate.

20. The microdriver system of claim 18, wherein the base includes at least one track, the arm configured to be translated relative to the base by sliding along the at least one track.
